(19) United States
(12) Patent Application Publication
(10) Pub. No.: US 2024/0239839 A1
(43) Pub. Date: Jul. 18, 2024

(54) PURIFICATION OF SPECIFIC TRIPEPTIDE BY USE OF ETHERS

(71) Applicant: DSM IP Assets B.V., Heerlen (NL)

(72) Inventors: Marc HEIDL, Kaiseraugst (CH); Thomas SCHAERER, Kaiseraugst (CH); Alexander SCHLIFKE-POSCHALKO, Kaiseraugst (CH)

(21) Appl. No.: 18/559,242

(22) PCT Filed: May 3, 2022
(86) PCT No.: PCT/EP2022/061791

§ 371 Date: Nov. 6, 2023

(30) Foreign Application Priority Data
May 10, 2021 (EP) 21172939.7

(51) Int. Cl. C07K 1/30; A61K 8/64; A61K 38/00; A61Q 19/00; C07K 1/34

(52) U.S. Cl. CPC C07K 1/30; A61K 8/64; A61Q 19/00; C07K 1/34; A61K 38/00

(57) ABSTRACT

The present invention relates to purification of a tripeptide of formula (I). By using said method, the tripeptide of formula (I) is obtained in a very pure form and due to this effect the purified tripeptide can be used in a variety of cosmetic and pharmaceutic compositions which are not accessible for the respective unpurified tripeptide.

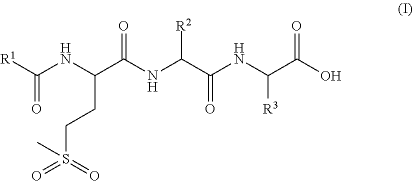

### TECHNICAL FIELD

The present invention relates to the purification of a tripeptide of the formula (I) and its use in cosmetic and pharmaceutical compositions.

### BACKGROUND OF THE INVENTION

Peptides, in general, play an important role in skin care in particular as anti-aging (anti-wrinkle, skin elasticity, contour remodeling) agents. However, many peptides are susceptible to oxidation and thus not stable when incorporated into a cosmetic composition.

The tripeptide of formula (I), as shown later-on in this document, is particularly well suited for cosmetic and pharmaceutical compositions.

This tripeptide of formula (I) (Ac-Met(O2)-Val-Val-OH) shows significant lower susceptibility against oxidation by atmospheric oxygen as compared to the corresponding tripeptide of the formula (I0) (Ac-Met-Val-Val-OH), as shown later-on in this document.

It has been shown that the tripeptide of formula (I) has an intense colour which severely limits its application in the field of cosmetics and pharmaceuticals. It has been found that this off-colour are due to impurities which are very difficult to remove by simple purification methods.

Attempts to find a suitable easy and economic method of purification have failed so far.

### SUMMARY OF THE INVENTION

Therefore, the problem to be solved by the present invention is to offer an economically favourable and efficient method of purification of compound of the formula (I).

Surprisingly, it has been found that the method of claim 1 offers a solution to this process.

It has been found that said process yield the tripeptide of the formula (I) without off-colour, i.e. that the tripeptide is white, in other words, colourless or at least essentially colourless, which is crucial for many applications, in such a degree as not yet known or expected.

It has been particularly found that there exist particularly two preferred embodiments of this method which offers the above mentioned advantages.

Due to this very efficient purification method, the tripeptide of the formula (I) can now be used also in cosmetic and pharmaceutic compositions which have been blocked up to now because the off-colour of tripeptide available until now.

Further aspects of the invention are subject of further independent claims. Particularly preferred embodiments are subject of dependent claims.

### DETAILED DESCRIPTION OF THE INVENTION

In a first aspect the present invention relates to a method of purification of a tripeptide of the formula (I)

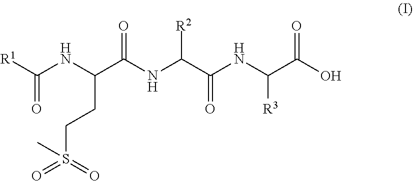

comprising the steps
a) providing a solution of a tripeptide of the formula (I) in a solvent which is selected from the group consisting of water, methanol, ethanol and acetic acid or mixtures thereof;
b) mixing an ether, particularly an ether selected from the group consisting of methyl tert-butyl ether, diethyl ether and diisopropyl ether or mixtures thereof with the solution of step a)
c) forming a precipitation of the compound of the formula (I);
d) separating the precipitated compound of the formula (I) formed in step c)
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
   either represent independently from each other a C₁₋₁₀-alkyl group, which are optionally substituted by up to three hydroxy groups; or represent together a C₂₋₁₀-alkylene group, which is optionally substituted by up to three hydroxy groups.

For sake of clarity, some terms used in the present document are defined as follows:

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂-CH₃ is considered as a C₄-alkyl group.

Analogously, a “C_{x-y}-alkylene” group is in the present document an alkylene group comprising x to y carbon atoms, i.e., for example, a C₂₋₃-alkylene group is an alkylene group comprising 2 to 3 carbon atoms. The alkylene group can be linear or branched. For example, —CH₂—CH₂—CH₂— and —CH(CH₃)—CH₂— and —C(CH₂—CH₃)— and —C(CH₃)₂— are all considered as a C₃-alkylene group.

An “aryl group” is an aromatic substituent. Preferred aryl groups are phenyl or naphthyl groups.

An “aralkyl” group is an alkyl group which is substituted by an aryl group. Accordingly, in the present document, a “C_{x-y}-aralkyl” group is an aralkyl group comprising x to y carbon atoms, i.e., for example, a C₇₋₁₆-aralkyl group is an aralkyl group comprising 7 to 16 carbon atoms. The aralkyl group can be linear or branched. For example, benzyl group (—CH₂—C₆H₅) is considered as a C₇-aralkyl group.

An “alkylaryl” group is an aryl group which is substituted by an alkyl group.

Accordingly, in the present document, a “C_{x-y}-alkylaryl” group is an alkylaryl group comprising x to y carbon atoms, i.e., for example, a C₇₋₁₆-alkylaryl group is an alkylaryl group comprising 7 to 16 carbon atoms. The alkylaryl group can be linear or branched. For example, the tolyl group (—C₆H₄CH₃) is considered as a C₇-alkylaryl group and the xylyl group (—C₆H₃(CH₃)₂) is considered as a C₈-alkylaryl group.

In case identical labels for symbols or groups are present in several formulae, in the present document, the definition of said group or symbol made in the context of one specific formula applies also to other formulae which comprises the same said label.

The term “independently from each other” in this document means, in the context of substituents, moieties, or groups, that identically designated substituents, moieties, or groups can occur simultaneously with a different meaning in the same molecule.

In the present document, any dotted line in formulae represents the bond by which a substituent is bound to the rest of a molecule.

In the present document any bond having dotted line ( [symbol] ) in a chemical formula represents independently from each other either a single carbon-carbon bond or a double carbon-carbon bond.

Any wavy line in any formula of in this document represents a carbon- carbon bond and which when linked to the carbon-carbon double bond is either in the Z or in the E-configuration. It is preferred in all molecules that the carbon-carbon double bond is in the E-configuration.

A “precipitation” as used in this document is a solid which is formed from a solution and separates from the liquid phase by the influence of gravity of earth, within a time of maximum 1 hours. Therefore, solids such as sols, gels or colloid dispersions, are not considered as precipitated in the sense of this document.

In step a) of the said process a solution of a tripeptide of the formula (I) is provided) in a solvent which is selected from the group consisting of water, methanol, ethanol and acetic acid or mixtures thereof.

Hence, also particularly binary or ternary or quaternary mixtures of these solvents can be used for this purpose such as, for example, water/acetic acid, methanol/ethanol, methanol/ethanol/water, water/methanol, water/ethanol, methanol/acetic acid, ethanol/acetic acid or methanol/ethanol/water/acetic acid.

For the purpose of this invention, it is important that a solution is prepared in step a). It is preferred that the solution is prepared so to assure that the concentration is as high as possible in formula (I).

It is preferred that the concentration of the tripeptide of the formula (I) in the solution of step a) is 5-30% by weight, preferably 15-20% by weight, relative to the weight of the solution.

It is preferred that the solution of tripeptide of the formula (I) in the solution of step a) is acidic, particularly having a pH of less than 6, particularly less than 5.

Acetic acid is particular preferred solvent used for step a).

Glacial acetic acid is a very preferred solvent used for step a).

The tripeptide is preferably prepared either by solid phase peptide synthesis using the respective FMOC-protected amino acids or by liquid phase peptide synthesis using the respective BOC-protected amino acids to form the tripeptide of the formula (I0), followed by an oxidation of the S-CH₃ group to the SO₂-CH₃ group by a suitable oxidizing agent.

The oxidation of the tripeptide of the formula (I0) to prepare the tripeptide of the formula (I) is particularly performed by hydrogen peroxide. Typically a stoichiometric excess of the oxidizing agent is used to assure quantitative formation of the tripeptide of the formula (I). The excess of oxidizing agent is preferably quenched, such as by using dimethyl sulfoxide (DMSO).

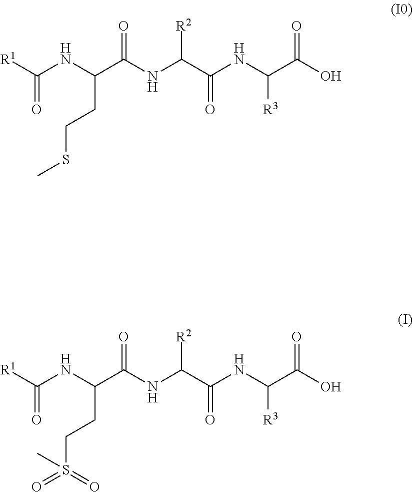

R¹ represents in one embodiment the tripeptide of the formula (I) a C₁₋₁₆-alkyl group which is optionally substituted by up to three hydroxy groups. R² and R³ represents in one of the embodiments a C₁₋₁₀-alkyl group which is optionally substituted by up to three hydroxy groups.

Preferably, the C₁₋₁₆-alkyl group or the C₁₋₁₀-alkyl group, which is optionally substituted by up to three hydroxy groups, is selected from the group consisting of methyl, ethyl, n-propyl, 1-methylethyl, 3-hydroxypropyl, 2,3-di-hydroxypropyl, 3-hydroxybutyl, 4-hydroxybutyl, n-butyl, 1-methylpropyl, 2-methyl-propyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethyl-propyl, 1-ethyl-1-methylpropyl, 1-ethyl-2-methylpropyl, 3,5,5-trimethylhexyl and 2,3-dihydroxypropyl groups, preferably methyl, ethyl, propyl, butyl, hexyl, heptyl, octyl or 2,3-hydroxypropyl group.

In another embodiment, R² and R³represent together a C₂₋₁₀-alkylene group, which is optionally substituted by up to three hydroxy groups. Preferably, the C₂₋₁₀-alkylene group, is selected from the group consisting of ethylene, propylene, butylene, pentylene and hexylene group.

Most preferably, the C₁₋₁₆-alkyl group and/or the C₁₋₁₀-alkyl group and/or the aryl group and/or the C₇₋₁₆-aralkyl group and or the C₇₋₁₆-alkylaryl group and/or the C₂₋₁₀-alkylene group are not substituted by any hydroxy groups.

It is preferred that in formula (I) R¹ represents a methyl group or H, and that R² and R³ represent both isopropyl groups.

It is most preferred that the compound of the formula (I) is the compound of the formula (I-A)

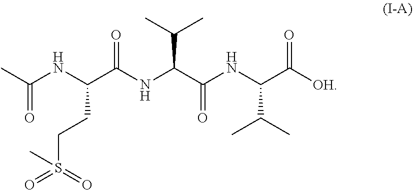 [SMILES: CC(=O)N[C@@H](CCS(C)(=O)=O)C(=O)N[C@H](C(=O)N[C@H](C(=O)O)C(C)C)C(C)C]

In step b) an ether, particularly an ether selected from the group consisting of methyl tert-butyl ether, diethyl ether and diisopropyl ether or mixtures thereof, is mixed with the solution of step a).

Hence, also particularly binary or ternary mixtures of these ethers can be used for this purpose such as, for example, methyl tert-butyl ether/diethyl ether or methyl tert-butyl ether/diisopropyl ether or methyl tert-butyl ether/diethyl ether/diisopropyl ether.

Methyl tert-butyl ether is a very preferred ether used for step b).

It is preferred that the ratio (volume/volume) of the solvent used in step a) to the ether used in step b) is 1/25 to 1/5, preferably 1/15 to 1/5, more preferably 1/12 to 1/8.

5 The mixing of the solution of step a) with the ether can be either that the solution of step a) is added to the ether or that the ether is added to the solution of step a). In a small scale (lab scale) it is preferred that the ether is added to the solution of step a) whereas in large scale (pilot or production scale) it is preferred that the solution of step a) is added to the ether.

It is further preferred, that the mixing in step b) is performed under stirring.

In step c) a precipitation of the compound of the formula (I) is formed. Step c) can take place after step b) or simultaneously during step b).

It has been shown, that particularly three preferred embodiment of the described process are particularly effective and cost-advantageous:

In a first of these preferred embodiments, the ether and the solution of step a) are mixed at temperatures significantly below the boiling point of the ether or the solvents used in step a), particularly at a temperature of between 30° C. and 0° C., preferably at about room temperature (25° C.). On mixing ether and solution of step a) a precipitation is formed, typically already during mixing.

In a second, and most preferred, of these preferred embodiments, after step a), the solution of step a) is heated to a temperature below the boiling point of the solvent used in step a).

By mixing the ether in step b) with the solution of step a), a precipitation of the tripeptide of the formula (I) is formed in step c).

It is further preferred that a step b″) is subsequently performed prior to the separation step d) 30

b″) cooling to a temperature below room temperature.

The precipitation in this embodiment is preferably formed in step c) during the cooling step b″).

It is much preferred that said cooling is not to a temperature below the freezing temperature of the solvents, respectively the mixtures thereof used the steps a) and b). Preferably, the cooling is a cooling to a temperature between 25° C. and 0°, particularly between 20° C. and 2° C., more preferred between 10° C. and 3° C.

In the third of these preferred embodiment, after step b), a step b′), followed by a step b″), is performed
b′) heating to a temperature below the boiling point of the solvent or solvent mixture used in step a) or the ether used in step b);
b″) cooling to a temperature below room temperature.

It is preferred that in step b′) the heating is preferably a heating to a temperature of between 50° C. and the boiling point of the solvent or solvent mixture. It is particularly preferred that the heating is a heating under reflux.

In step c) a precipitation is formed. It is important to stress that this precipitation separates from the liquid phase by the influence of gravity of earth, within a time of maximum 1 hours. It is preferred that the precipitation is in the form of particles of a mean diameter of more than 1 micrometre, preferably more than 10 micrometre. It is preferred that the mean diameter of said particles are in the range of between 1 μm and 100 μm, particularly between 5 μm and 50 μm, more particularly between 5 μm and 30 μm. The precipitation can be an amorphous or crystalline form. Preferably, the precipitated tripeptide of the formula (I) is in crystalline form.

In step d) the precipitated tripeptide of the formula (I) formed in step c) is separated. The separation is performed preferably by means of filtration, more preferably by a filtration over a Nutsche filter or a Büchner funnel or an agitated nutsche filter (ANF) or a glass frit (sintered glass) filter. Preferably, for this separation step d) a vacuum is applied for the filtration.

Preferably, for this separation step d) a vacuum is applied for the filtration.

If needed or desired, the tripeptide of the formula (I), separated in step d) can be subjected to one or more additional purification cycles comprising steps a) to d).

Typically, additional purification cycles are not needed as the process provides the tripeptide of the formula (I) in a very pure form after the (first) step d).

It has been found that the above process is able to provide the tripeptide of the formula (I) in an extraordinary highly pure form.

The so purified tripeptide of the formula (I) is particularly advantageous as it has no off-colour, i.e. that the tripeptide is white, in other words, colourless or at least essentially colourless, which is crucial for many applications of the tripeptide.

In a very preferred application, the tripeptide of the formula (I), purified by the process as described above is its use in cosmetics or pharmaceuticals and their manufacturing.

Hence, in a further aspect, the present invention relates to a cosmetic or pharmaceutical composition comprising a tripeptide of the formula (I) which has been purified by a method as described above in great details.

The term ‘cosmetic composition’ refers to compositions which are used to treat, care for or improve the appearance of the skin and/or the scalp. Particular advantageous cosmetic compositions according to the present invention are skin care compositions.

The cosmetic or pharmaceutical compositions according to the invention are preferably intended for topical application, which is to be understood as the external application to keratinous substances, such as in particular the skin.

The term ‘cosmetically acceptable carrier’ as used herein refers to a physiologically acceptable medium which is compatible with keratinous substances. Suitable carriers are well known in the art and are selected based on the end-use application. Preferably, the carriers of the present invention are suitable for application to skin (e.g., sunscreens, creams, milks, lotions, masks, serums, hydrodispersions, foundations, creams, creamgels, or gels etc.). Such carriers are well-known to one of ordinary skill in the art, and can include one or more compatible liquid or solid filler, diluent, excipient, additive or vehicle which are suitable for application to skin. Particularly preferred cosmetically acceptable carrier is selected from the group consisting of water, oils, fats, waxes, organic solvents and fillers. The exact amount of carrier will depend upon the level of the compound of formula (I) and any other optional ingredients that one of ordinary skill in the art would classify as distinct from the carrier (e.g., other active components). The compositions of the present invention preferably comprise from about 75% to about 99.999%, more preferably from about 85% to about 99.99%, still more preferably from 90% to about 99%, and most preferably, from about 93% to about 98%, by weight of the composition, of a carrier.

The cosmetic or pharmaceutical compositions of the present invention can be formulated into a wide variety of product types, including creams, waxes, pastes, lotions, milks, mousses, gels, oils, tonics, and sprays. Preferably the compounds of formula (I) are formulated into lotions, creams, gels, and tonics. These product forms may be used for a number of applications, including, but not limited to, hand and body lotions, facial moisturizers, anti-ageing preparations, make-ups including foundations, and the like. Any additional components required to formulate such products vary with product type and can be routinely chosen by a person skilled in the art.

If compositions of the present invention are formulated as an aerosol and applied to the skin as a spray-on product, a propellant is added to the composition.

The cosmetic or pharmaceutical compositions according to the present invention can be prepared by conventional methods in the art such as e.g. by admixing a compound of formula (I) with all the definitions and preferences given herein with the cosmetically acceptable carrier. The cosmetic compositions of the invention (including the carrier) may comprise further conventional cosmetic adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/ or sequestering agents, essential oils, skin sensates, astringents, pigments or any other ingredients usually formulated into such compositions.

In accordance with the present invention, the cosmetic or pharmaceutical compositions according to the invention may also comprise further cosmetically active ingredients conventionally used in cosmetic compositions. Exemplary active ingredients encompass further self-tanning agents, UV-filters, agents for the treatment of hyperpigmentation; agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the active ingredients as well as the cosmetic excipients, diluents, adjuvants, additives etc. can, based on the desired product form and application, easily be determined by the person skilled in the art. The additional ingredients can either be added to the oily phase, the aqueous phase or separately as deemed appropriate.

The cosmetically active ingredients useful herein can in some instances provide more than one benefit or operate via more than one mode of action.

Of course, a person skilled in the art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

These compositions can be used particularly for the prevention, treatment and/or reduction of wrinkles, improvement of skin elasticity and/ or for contour remodeling. The compositions are particularly brought in contact to a skin in need of such a treatment with a compound of formula (I) with all the definitions and preferences as given herein and optionally appreciating the effect.

Therefore, the invention relates, in a further aspect, to a process of manufacturing a cosmetic or pharmaceutical composition comprising the steps d
i) purifying a tripeptide of the formula (I) by a method as described above in great detail yielding a purified tripeptide of the formula (I);
ii) providing a cosmetically acceptable carrier
iii) combining the purified tripeptide of the formula (I) of step i) with the cosmetically acceptable carrier of step ii) and with optionally further ingredients to yield a cosmetic or pharmaceutical composition.

Accordingly, as described above, said process of manufacturing yields a cosmetic or pharmaceutical composition. Therefore, a further aspect of the present invention relates to a cosmetic or pharmaceutical composition, particularly a cosmetic composition, which is produced according to a said process.

As shown above the tripeptide of the formula (I), purified by the process as described above, can be used in a cometic composition.

Hence, in a further aspect, the present invention relates to a use of a tripeptide of the formula (I), which is purified using a method as described above in great details in a cometic composition.

### EXAMPLES

The present invention is further illustrated by the following experiments.

### Synthesis of Tripeptide of the Formula (I-A)

The tripeptide Ac-Met-Val-Val-OH has been prepared by solid phase peptide synthesis using FMOC-protected amino acids from methionine (Met) and valine (Val):

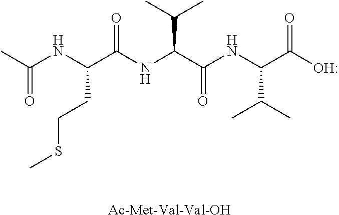

The tripeptide Ac-Met-Val-Val-OH has then been treated with H2O2 an stochiometric excess followed by quenching with DMSO to yield the tripeptide of the formula (I-A) (Ac-Met(O2)-Val-Val-OH) which has been isolated and used for the following examples

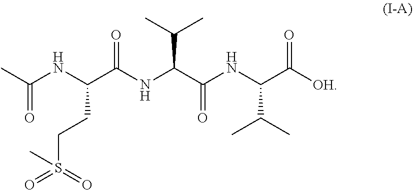 [SMILES: CC(=O)N[C@@H](CCS(C)(=O)=O)C(=O)N[C@H](C(=O)N[C@H](C(=O)O)C(C)C)C(C)C]

### First Series of Examples

213 mg Ac-Met(O2)-Val-Val-OH has been dissolved in 1.05 ml glacial acetic acid at 23° C. To this clear solution 10 ml of the solvent as given in table 1 have been added.

TABLE 1
Different solvents used in step b).

| Ex. | Solvent added | Observation |
|---|---|---|
| 1 | Methyl tert-butyl ether | Precipitation in fine particles |
| 2 | Diisopropyl ether | Precipitation in fine particles |
| Ref. 1 | No additional solvent | Remains clear solution |
| Ref. 2 | Ethanol | Remains clear solution |
| Ref. 3 | Methanol | Remains clear solution |
| Ref. 4 | Acetonitrile | Remains clear solution |

In the examples 1 and 2 a precipitate of the tripeptide in fine particles is formed at the bottom of the vessel which is separated by filtration over a glass frit filter. In the comparative examples Ref.1 to Ref.4 no precipitation has been formed even when further adding the respective solvent and, hence, no solid tripeptide could be separated or isolated.

### Second Series of Examples

Ac-Met(O2)-Val-Val-OH in an amount given in table 2 has been dissolved in 0.5 ml glacial acetic acid at 70° C. To this clear solution, 10 ml of the solvent as given in table 2 have been added and cooled to 4° C.

TABLE 2
Different solvents used in step b) mixed with the solution at 70° C.

| | (I-A) | | |
|---|---|---|---|
| Ex. | [mg] | Solvent added | Observation |
| 3 | 213 | Methyl tert-butyl ether | Precipitation in fine particles |
| 4 | 213 | Diisopropyl ether | Precipitation in fine particles |
| Ref. 5 | 101 | No additional solvent | Remains clear solution |
| Ref. 6 | 210 | Ethanol | Remains clear solution |
| Ref. 7 | 185 | Methanol | Remains clear solution |
| Ref. 8 | 114 | Butanol | Remains clear solution |
| Ref. 9 | 211 | Acetonitrile | Remains clear solution |

In the examples 3 and 4, a precipitates of the tripeptide in fine particles is formed at the bottom of the vessel which are separated by filtration over a glass frit filter. In the comparative examples Ref.5 to Ref.9 even at prolonged storage at 4° C., any precipitation has been formed and, hence, no solid tripeptide could be separated or isolated.

### Example Ref.10

243 mg Ac-Met(O2)-Val-Val-OH has been added to different amounts of methyl tert-butyl ether. It was not possible to obtain a solution of the tripeptide (prerequisite for steps a), b) and c)) even at prolonged time at a heating at reflux temperature.

### Example Ref.11

243 mg Ac-Met(O2)-Val-Val-OH has been dissolved in 3.6 ml ethanol at reflux temperature. Upon cooling the solution forms a gel which does not form any particles and could not be separated.

1. A method of purification of a tripeptide of the formula (I) 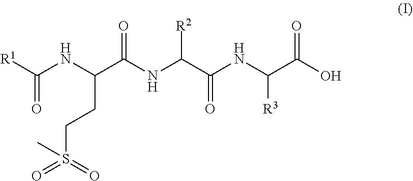
comprising the steps
a) providing a solution of a tripeptide of the formula (I) in a solvent which is selected from the group consisting of water, methanol, ethanol and acetic acid or mixtures thereof;
b) mixing an ether, particularly an ether selected from the group consisting of methyl tertbutyl ether, diethyl ether and diisopropyl ether or mixtures thereof, with the solution of step a)
c) forming a precipitation of the compound of the formula (I);
d) separating the precipitated compound of the formula (I) formed in step c)
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.

2. The method according to claim 1, characterized in that
R¹ is methyl or H,
R² and R³ are both isopropyl.

3. The method according to claim 1, characterized in that the compound of the formula (I) is the compound of the formula (I-A)

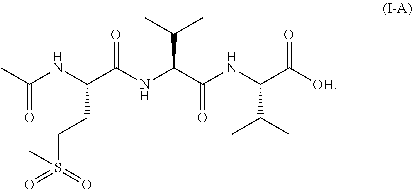 [SMILES: CC(=O)N[C@@H](CCS(C)(=O)=O)C(=O)N[C@H](C(=O)N[C@H](C(=O)O)C(C)C)C(C)C]

4. The method according to claim 1, wherein after step a) the solution is heated to a temperature below the boiling point of the solvent used in step a).

5. The method according to claim 1, wherein after step b), a step b′), followed by step b″), is performed
b′) heating to a temperature below the boiling point of the solvent or solvent mixture used in step a) or the ether used in step b);
b″) cooling to a temperature below room temperature.

6. The method according to claim 1, wherein the solvent used in step a) is acetic acid.

7. The method according to claim 1, wherein the solvent used in step a) is glacial acetic acid.

8. The method according to claim 1, wherein ether used in step b) is methyl tert-butyl ether.

9. The method according to claim 1, wherein separation of the precipitated compound of the formula (I) is performed in step d) by means of filtration.

10. The method according to claim 1, wherein the concentration of the tripeptide of the formula (I) in the solution of step a) is 5-30% by weight, preferably 15-20% by weight, relative to the weight of the solution.

11. The method according to claim 1, wherein the ratio (volume/volume) of the solvent used in step a) to the ether used in step b) is 1/25 to 1/5, preferably 1/15 to 1/5, more preferably 1/12 to 1/8.

12. The method according to claim 1, wherein the tripeptide of the formula (I) is prepared from the tripeptide of the formula (I0) by oxidation, particularly by oxidation with H₂O₂,

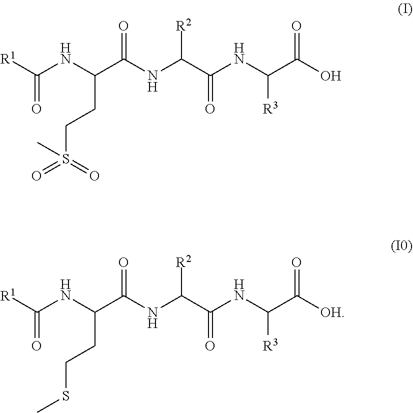

13. A process of manufacturing a cosmetic or pharmaceutical composition comprising the steps
i) purifying a tripeptide of the formula (I) by a method according to claim 1, yielding a purified tripeptide of the formula (I);
ii) providing a cosmetically acceptable carrier
iii) combining the purified tripeptide of the formula (I) of step i) with the cosmetically acceptable carrier of step ii) and with optionally further ingredients to yield a cosmetic or pharmaceutical composition.

14. A cosmetic or pharmaceutical composition which is produced according to a process according to claim 13.

15. A cosmetic or pharmaceutical composition comprising a tripeptide of the formula (I)
which has been purified by a method according to claim 1, and a cosmetically acceptable carrier 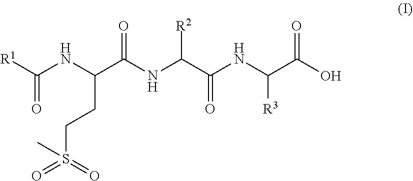
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.

16. Use of a tripeptide of the formula (I), which is purified using a method according to claim 1, in a cometic composition 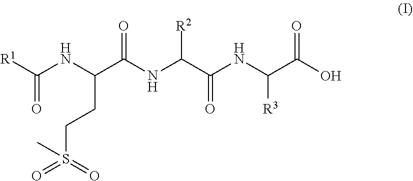
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.
